(19) United States
(12) Patent Application Publication
(10) Pub. No.: US 2015/0305656 A1
(43) Pub. Date: Oct. 29, 2015

(54) Motor Function Measurement Apparatus, Method and Recording Medium Storing Program

(71) Applicant: TANITA CORPORATION, Tokyo (JP)

(72) Inventors: Tomoko Takehara, Tokyo (JP); Nobuya Sakai, Tokyo (JP); Yoshio Sakai, Tokyo (JP)

(73) Assignee: TANITA CORPORATION, Tokyo (JP)

(21) Appl. No.: 14/691,806

(22) Filed: Apr. 21, 2015

(30) Foreign Application Priority Data
Apr. 23, 2014 (JP) 2014-089161

(51) Int. Cl. A61B 5/11; A61B 5/00

(52) U.S. Cl. CPC A61B 5/1124; A61B 5/1116; A61B 5/7278

(57) ABSTRACT

A motor function measurement apparatus that acquires measurement data including at least one of an acceleration or a trunk inclination angle of a user, the measurement data being measured during a measurement period in which the user performs a predetermined action, and that, based on the measurement data, calculates a motor function evaluation value related to a motor function of the user.

### CROSS-REFERENCE TO RELATED APPLICATION

This application is based on and claims the benefit of priority of the prior Japanese Patent Application No. 2014-089161, filed on Apr. 23, 2014, the entire contents of which are incorporated herein by reference.

### BACKGROUND

1. Technical Field

The present invention relates to a motor function measurement apparatus, method, and program.

2. Related Art

In order to preserve and increase physical strength at an advanced age, and to lead a healthy and meaningful life, there is a need to ascertain one's state of physical strength that is the foundation of such and a need to appropriately exercise according to the ascertained state of physical strength.

Hitherto, physical strength testing has served as a method for ascertaining motor functions such as muscle strength, balance ability, and mobility. Examples of measurements of muscle strength include measurement of grip, and measurement of leg extension force. Examples of measurements of balance ability include measuring the time spent standing on one leg with the eyes open, and measurement of functional reach. Examples of measurements of mobility include Time Up and Go (TUG) testing, and walking speed measurements.

Patent Document 1 describes a technology for measuring motor function by causing a subject to change their standing posture, and computing parameters of postural stability.

An evaluation method using a chart of pain related questions, and an evaluation method using a visual analogue scale (VAS) serve as methods of evaluation of a degree of physical pain.

### Patent Documents

Patent Document 1: Japanese Patent No. 4925284

However, the conventional technology mentioned above has various issues such that equipment for measurements is expensive, a complex manipulation is required, measurements by oneself and interpreting the results thereof are difficult, a high level of analytical skill is required, places for measurements are restricted, and it takes time to do the measurements.

For people of advanced age in particular, motor function sometimes deteriorates rapidly. Thus, ascertaining motor function deterioration using occasional motor function measurements alone is difficult, while, performing regular motor function measurements in everyday life using conventional technology is difficult due to the problems thereof as stated above.

### SUMMARY

An object of the present invention is to provide a motor function measurement apparatus, method, and program that enable easy measurement of motor function.

In order to solve the above problems, a motor function measurement apparatus includes: a measurement data acquisition section that acquires measurement data including at least one of an acceleration or a trunk inclination angle of a user, the measurement data being measured during a measurement period in which the user performs a predetermined action; and a motor function evaluation value computation section that, based on the measurement data, calculates a motor function evaluation value related to a motor function of the user.

Configuration may be made such that the measurement data includes the acceleration and the trunk inclination angle, and the motor function evaluation value computation section calculates, based on the acceleration and the trunk inclination angle of the user measured during the measurement period, a motor function index related to muscle strength and balance ability of the user as the motor function evaluation value.

Configuration may also be made such that the motor function measurement apparatus further includes an age acquisition section that acquires age data related to an age of the user, and the motor function evaluation value computation section calculates, based on the acceleration, the trunk inclination angle and the age data, a motor function age related to muscle strength and balance ability of the user as the motor function evaluation value.

Configuration may also be made such that the measurement data includes the acceleration, and the motor function evaluation value computation section calculates, based on the acceleration of the user measured during the measurement period, a strength score related to muscle strength of the user as the motor function evaluation value.

Configuration may also be made such that the motor function evaluation value computation section calculates, based on a maximum value of acceleration of the user measured during the measurement period, a difference between a minimum value and the maximum value of acceleration of the user measured during the measurement period, and a maximum rate of change in the acceleration of the user measured during the measurement period, a strength score related to the muscle strength of the user as the motor function evaluation value.

Configuration may also be made such that the measurement data includes the trunk inclination angle, and the motor function evaluation value computation section calculates, based on the trunk inclination angle of the user measured during the measurement period, an angle score related to a balance ability of the user as the motor function evaluation value.

Configuration may also be made such that the motor function evaluation value computation section calculates, based on a difference between a maximum value of the trunk inclination angle of the user measured during the measurement period and a reference trunk inclination angle that is a trunk angle of the user measured during a rest period of the measurement period in which the user is in a resting state, and based on a number of peaks in the trunk inclination angle of the user measured during the measurement period, an angle score related to the balance ability of the user as the motor function evaluation value.

Configuration may also be made such that the measurement data includes the trunk inclination angle of the user in a left-right direction of the user, and the motor function measurement apparatus further includes a determination section that determines whether or not pain arises at either of a left side or a right side of a body of the user based on the trunk inclination angle of the user in the left-right direction measured during the measurement period.

Configuration may also be made such that the motor function measurement apparatus further includes an output section that outputs the motor function evaluation value, and outputs advice data corresponding to the motor function evaluation value.

Configuration may also be made such that the predetermined action is an action of standing up from a seated state in a chair.

Configuration may also be made such that the motor function measurement apparatus further includes a measurement section that measures at least one of the acceleration or the trunk inclination angle.

A motor function measurement method includes: acquiring measurement data including at least one of an acceleration or a trunk inclination angle of a user, the measurement data being measured during a measurement period in which the user performs a predetermined action; and based on the measurement data, computing a motor function evaluation value related to a motor function of the user.

A motor function measurement program causes a computer to execute a process. The process includes: acquiring measurement data including at least one of an acceleration or a trunk inclination angle of a user, the measurement data being measured during a measurement period in which the user performs a predetermined action; and based on the measurement data, computing a motor function evaluation value related to a motor function of the user.

### Advantageous Effects of Invention

The present invention exhibits advantageous effects such as enabling easy measurement of motor function.

### BRIEF DESCRIPTION OF DRAWINGS

Embodiments of the invention will be described in detail with reference to the following figures, wherein:

FIG. 1 is a block diagram of a motor function measurement apparatus;

FIG. 2 is an exterior view of a motor function measurement apparatus;

FIG. 3 is a flowchart of processing according to a motor function measurement program;

FIG. 4 is an explanatory diagram for explaining computation of a trunk inclination angle;

FIG. 5 is a waveform diagram of composite acceleration and a trunk inclination angle;

FIG. 6 is a diagram for explaining the number of peaks in a trunk inclination angle;

FIG. 7 is a diagram illustrating an example of a waveform diagram of composite acceleration and trunk inclination angle;

FIG. 8 is a diagram illustrating an example of a waveform diagram of composite acceleration and trunk inclination angle;

FIG. 9 is a diagram for explaining a standing up action;

FIG. 10 is a diagram illustrating an example of a display of motor function evaluation values;

FIG. 11 is a diagram illustrating an example of a display of motor function evaluation values;

FIG. 12 is diagram illustrating an example of a display of motor function evaluation values;

FIG. 13 is a diagram illustrating a state in which a body is inclined in a front-rear direction;

FIG. 14 is a diagram illustrating a state in which a body is inclined in a left-right direction;

FIG. 15 is a diagram illustrating an example of waveforms of inclination angles θx in a left-right direction; and

FIG. 16 is a block diagram of a configuration in which a measurement device is connected to a personal computer.

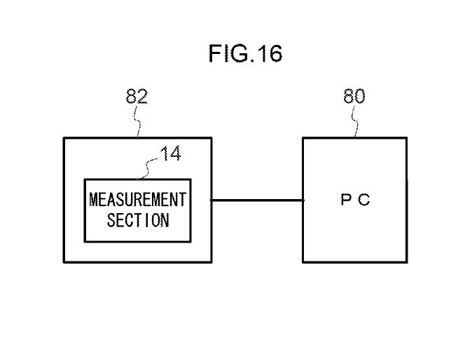

### DESCRIPTION OF EMBODIMENTS

Explanation follows regarding an exemplary embodiment of the present invention.

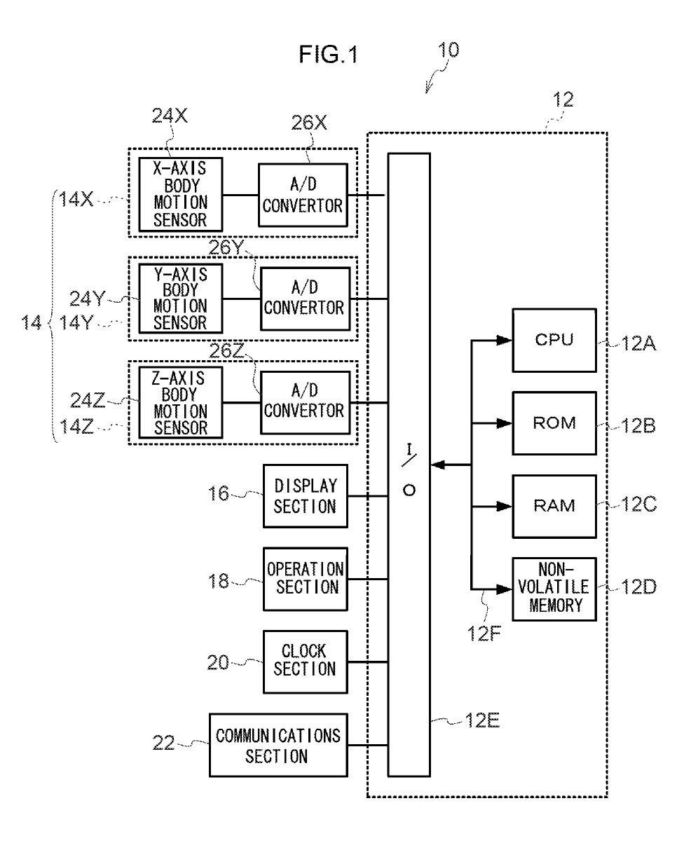

FIG. 1 is a block diagram of a motor function measurement apparatus 10 according to the present exemplary embodiment. As illustrated in FIG. 1, the motor function measurement apparatus 10 includes a controller 12, a measurement section 14, a display section 16, an operation section 18, a clock section 20, and a communications section 22. The measurement section 14 includes an x-axis acceleration detection section 14X, a y-axis acceleration detection section 14Y, and a z-axis acceleration detection section 14Z.

The controller 12 is configured by a central processing unit (CPU) 12A, read only memory (ROM) 12B, random access memory (RAM) 12C, non-volatile memory 12D, and an input/output interface (I/O), connected to one another via a bus 12F. In this case, a motor function measurement program that causes the CPU 12A of the controller 12 to execute motor function measurement processing, described below, is, for example, written to the non-volatile memory 12D, and the motor function measurement program is read and executed by the CPU 12A. The motor function measurement program may be provided on a recording medium such as a CD-ROM or a memory card, or may be downloaded from a server, not illustrated in the drawings.

The I/O 12E is connected to the x-axis acceleration detection section 14X, the y-axis acceleration detection section 14Y, the z-axis acceleration detection section 14Z, the display section 16, the operation section 18, the clock section 20, and the communications section 22.

The x-axis acceleration detection section 14X includes an x-axis body motion sensor 24X that, among the mutually orthogonal x-axis, y-axis, and z-axis, detects acceleration in the direction of the x-axis (referred to as x-axis acceleration hereafter), and an A/D convertor 26X that converts the x-axis acceleration detected by the x-axis body motion sensor 24X into digital data.

The y-axis acceleration detection section 14Y includes an y-axis body motion sensor 24Y that detects acceleration in the direction of the y-axis (referred to as y-axis acceleration hereafter), and an A/D convertor 26Y that converts the y-axis acceleration detected by the y-axis body motion sensor 24Y into digital data.

The z-axis acceleration detection section 14Z includes an z-axis body motion sensor 24Z that detects acceleration in the direction of the z-axis (referred to as z-axis acceleration hereafter), and an A/D convertor 26Z that converts the z-axis acceleration detected by the z-axis body motion sensor 24Z into digital data.

As the display section 16, for example, a liquid crystal panel or the like may be used. Various screen image, such as various setting screen images, measurement result screens, and the like are displayed on the display section 16.

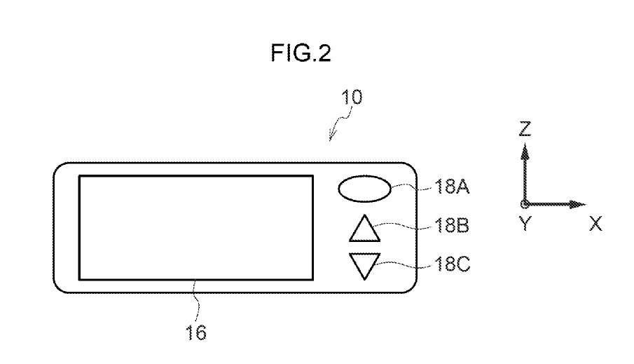

The operation section 18 is an operation section for performing various operations such as input operations for user data, and others. An example of exterior view of the motor function measurement apparatus 10 is illustrated in FIG. 2. As illustrated in FIG. 2, the operation section 18 is configured including plural operation buttons 18A to 18C. As an example, the operation button 18A functions as a button for turning the power supply of the motor function measurement apparatus 10 on/off, and for performing confirmation operations in various screen images. The operation buttons 18B, 18C function as buttons for scrolling the screen images up and down, or for moving a cursor up and down on the screen images.

The display section 16 and the operation section 18 may be a touch panel, and may be configured so as to be operable by directly touching the screen image.

The clock section 20 includes a function that acquires the current time, and a timer function that times a set time period.

The communications section 22 includes a communications function that sends/receives data to/from an external device using wireless communication or wired communication. The motor function measurement apparatus 10 is thereby capable of communication with, for example, an external device such as a personal computer, a mobile phone, a smartphone, or a tablet terminal.

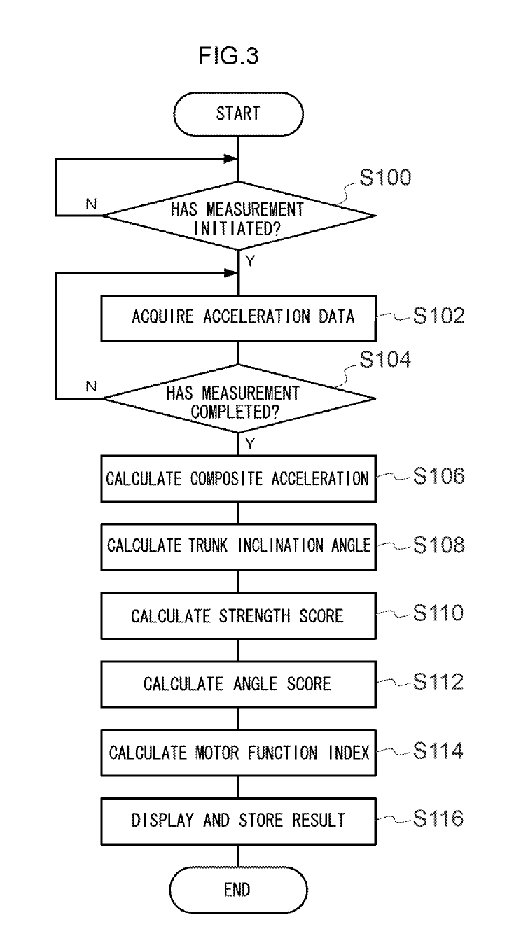

Next, as an example of operation of the present exemplary embodiment, explanation follows regarding processing executed by the CPU 12A of the controller 12 based on the motor function measurement program, with reference to the flowchart illustrated in FIG. 3. The processing illustrated in FIG. 3 is executed when execution of the motor function measurement program is instructed by the user operating the operation section 18 of the motor function measurement apparatus 10.

In the present exemplary embodiment, explanation follows regarding computing a motor function evaluation value related to motor functions of a user such as a person of advanced age, based on acceleration detected when the user wares the motor function measurement apparatus 10 at an upper portion of the navel, and performs an action of standing up from a seated state in a chair as a predetermined action. When the motor function measurement apparatus 10 is worn at the upper portion of the navel, the apparatus is to be worn such that the z-axis runs along an upright direction, as illustrated, for example, in FIG. 2, if the user is upright. The region in which the motor function measurement apparatus 10 is worn is not limited to the upper portion of the navel, and it may also be worn on the head or chest. Namely, any region, usually on the upper half of body, from which the inclination angle of the trunk can be detected when standing up from a chair is appropriate.

First, the user operates the operation section 18 of the motor function measurement apparatus 10 while in a seated state in a chair, and instructs measurement initiation.

At step S100 determination is made as to whether or not measurement initiation has been instructed by the user operating the operation section 18. Processing transitions to step S102 if measurement initiation was instructed, and processing stands by until measurement initiation is instructed if measurement initiation has not been instructed. In computation of an angle score, described later, because the angle score is calculated based on an average value of trunk inclination angle detected in a predetermined rest period (for example, several seconds) that the user spends in resting state sitting in a chair, the user must spend the predetermined rest period, which starts from when the user instructs measurement initiation, and be at rest without immediately standing up. A message may therefore be displayed on the display section 16, instructing the user to rest without immediately standing up, from sitting in the chair until the passing of the predetermined rest period.

At step S102, the x-axis acceleration, the y-axis acceleration, and the z-axis acceleration detected by the x-axis acceleration detection section 14X, the y-axis acceleration detection section 14Y, and the z-axis acceleration detection section 14Z, respectively, are acquired.

At step S104, determination is made as to whether or not the measurement has completed. Processing transitions to step S106 if the measurement has completed, and processing returns to step S102 and continues acquisition of the x-axis acceleration, the y-axis acceleration, and the z-axis acceleration if the measurement is continuing. Whether or not the measurement has completed may be determined according to whether or not measurement completion has been instructed by the user operating the operation section 18, or may be determined according to whether or not a measurement time, preset based on an estimated time period from the user instructing measurement initiation until performing a standing up action from the seated state in the chair, has elapsed.

At step S106, a composite acceleration is calculated for respective time points based on the x-axis acceleration, y-axis acceleration, and z-axis acceleration at respective time points measured in the measurement period from measurement initiation to measurement completion. Specifically, the composite acceleration is calculated by computing the root-mean-square of the x-axis acceleration, the y-axis acceleration, and the z-axis acceleration. Namely, the composite acceleration V is calculated according to the following equation, wherein ax is the x-axis acceleration, ay is the y-axis acceleration, and az is the z-axis acceleration.

V=\sqrt{ax²+ay²+az²} (1)

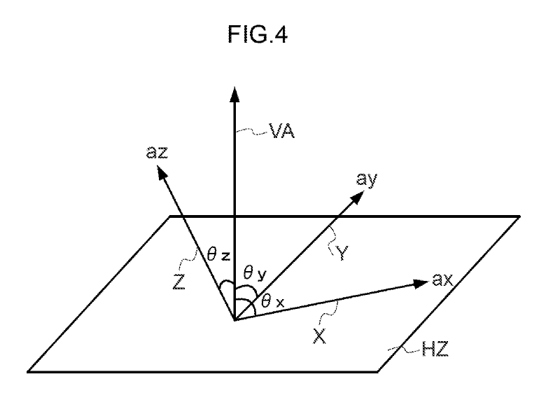

At step S108, the trunk inclination angle is calculated for each time point measured during the measurement period from measurement initiation to measurement completion. As illustrated in FIG. 4, an axis orthogonal to a horizontal plane HZ serves as a vertical axis VA, and the x-axis, the y-axis, and the z-axis, have inclination angles with respect to the vertical axis VA of θx, θy, and θz respectively, which can be calculated using the following equations, in which g is gravitational acceleration.

θx=cos⁻¹(ax/g) (2)

θy=cos⁻¹(ay/g) (3)

θz=cos⁻¹(az/g) (4)

In the present exemplary embodiment, since the motor function measurement apparatus 10 is worn such that the z-axis is along the upright direction when the user is upright as described above, the inclination angle θz of the z-axis is calculated as the trunk inclination angle θ of the user using Equation (4).

At step S110, a strength score PO related to the muscle strength of the user is calculated based on the composite acceleration V calculated at step S106, using the following equation.

PO=a1×peak_max+b1×Range+c1×jerk (5)

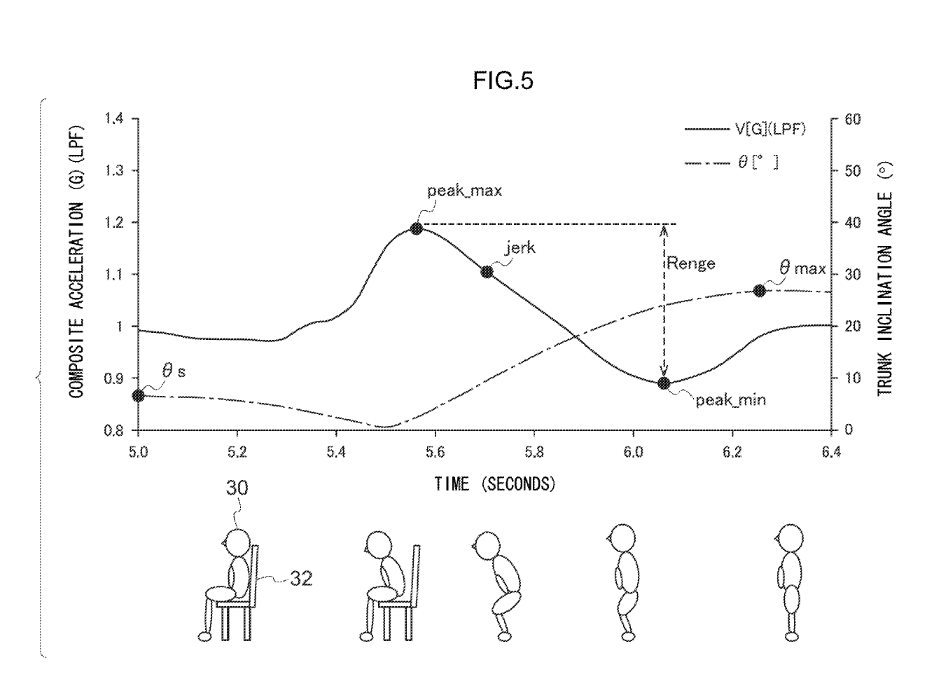

Herein, peak_max is the maximum value of the composite acceleration V measured in the measurement period, as illustrated in FIG. 5. As an example, the measurement period in FIG. 5 is 6.4 seconds. And, in FIG. 5, the waveforms of the composite acceleration V and the trunk inclination angle θ for the rest period of the measurement period (5 seconds as an example in the present exemplary embodiment), spent by the user resting sitting in a chair 32 are omitted. Moreover, Range is the difference between the maximum value peak_max, and the minimum value peak_min of the composite acceleration V measured during the measurement period. Moreover, jerk is the maximum rate of change of the composite acceleration V (maximum value of the jerk, i.e., the change rate of acceleration with respect to time)) measured during the measurement period.

In the exemplary case illustrated in FIG. 5, the rate of change in the composite acceleration V reaches a maximum in the vicinity of 5.7 seconds, namely, when the buttocks of a user 30 depart from the chair 32. In other words, the point where the rate of change in the composite acceleration V reaches a maximum can be said to be such a point in time when the thighs of the user 30 exert muscular force, moving the center of gravity. The strength score PO calculated using equation (5), in which peak_max that is the maximum value of the composite acceleration V, Range that is the difference between peak_max, the maximum value of the composite acceleration V and peak_min, the minimum value of the composite acceleration V, and jerk, that is the maximum rate of change in the composite acceleration V are parameters, can therefore be said to be an appropriate motor function evaluation value representing the muscle strength of the user 30. Here, a1, b1, and c1 are coefficients determined by using such a technique as principle component analysis or linear regression, for example on measurement results, of the composite acceleration V for several subjects performing a standing up action from a seated state in a chair, and are set to values such that the higher is the muscle strength of the user, the higher is the strength score PO.

At step S112, an angle score TR is calculated based on the trunk inclination angles θ at each time point calculated at step S108, using the following equations.

TR=a2×θ1+b2×peak_num (6)

θ1=θmax−θs (7)

Here, θmax is the maximum value of the trunk inclination angle θ among those measured at respective time points during the measurement period. Moreover, θs is a reference trunk inclination angle, and is an average value of the trunk inclination angles θ that, out of the trunk inclination angles θ measured at respective time points during the measurement period, were measured during the rest period (5 seconds as an example in the present exemplary embodiment). FIG. 5 illustrates an exemplary case where the trunk inclination angle θ that was 5 seconds after the measurement initiation corresponds to the reference trunk inclination angle θs. The reference trunk inclination angle θs may be a value representing a typical value of the trunk inclination angle θ during the rest period, and may, for example, be the median value. Moreover, the rest period is not limited to 5 seconds.

The reason why the trunk inclination angle θ1 used in the computation of the angle score TR is set to a value calculated by subtracting the reference trunk inclination angle θs from the maximum value of the trunk inclination angle θ, is to compute the angle score TR with high accuracy by canceling an effect caused in such a case where the trunk of the user 30 is inclined even in the state sitting in the chair and being at rest.

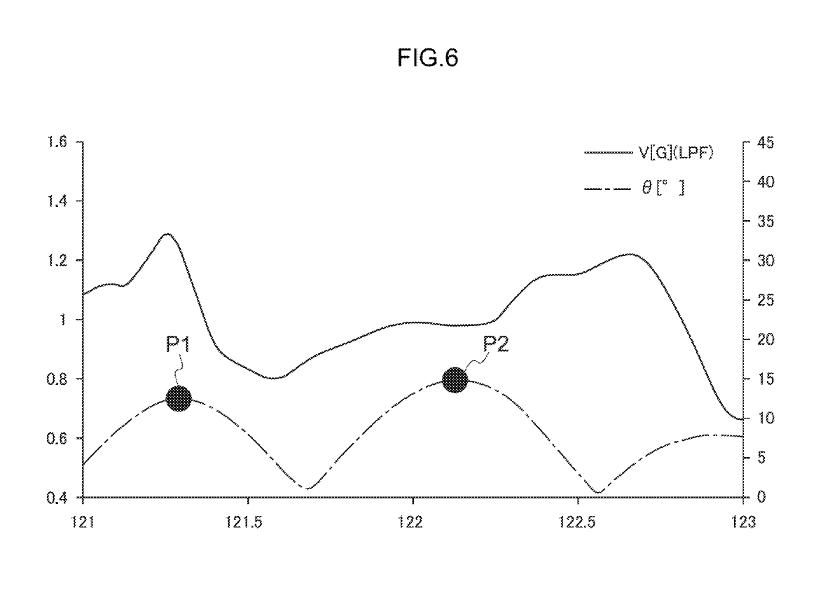

The number of peaks that appear in the waveform of the trunk inclination angle θ during the measurement period corresponds to peak_num. For example, if peaks appear in the waveform of the trunk inclination angle θ at the two points, such as the peaks of p1, p2 illustrated in FIG. 6, then peak_num=2.

Since the user 30 rises to his/her feet maintaining his/her balance by inclining his/her trunk forward and moving the position of his/her center of gravity to the sole when the user 30 stands up from the chair 32, the angle score TR calculated using Equation (6), using the trunk inclination angle θ and the number of peaks in the waveform of the trunk inclination angle θ as parameters, can be said to be a motor function evaluation value representing standing balance performance of the user 30. Since the trunk becomes inclined further forward when rising to one's feet when there is pain in the knees or waist, the degree of pain can also be ascertained from the angle score TR. Moreover, a2, and b2 are coefficients determined by using a such technique as principle component analysis or linear regression, for example, on measurement results, of the trunk inclination angle θ for several subjects performing a standing up action from a seated state in a chair, and are set to values such that the higher is the standing balance ability of the user 30, the higher is the angle score TR.

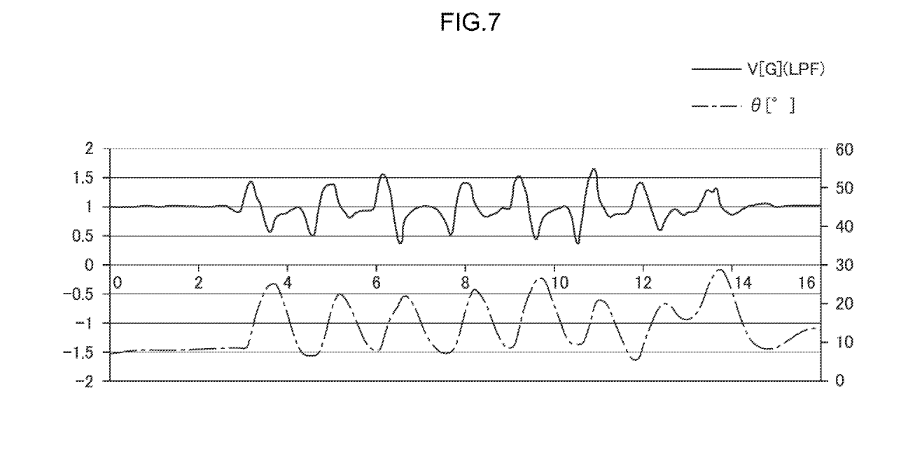

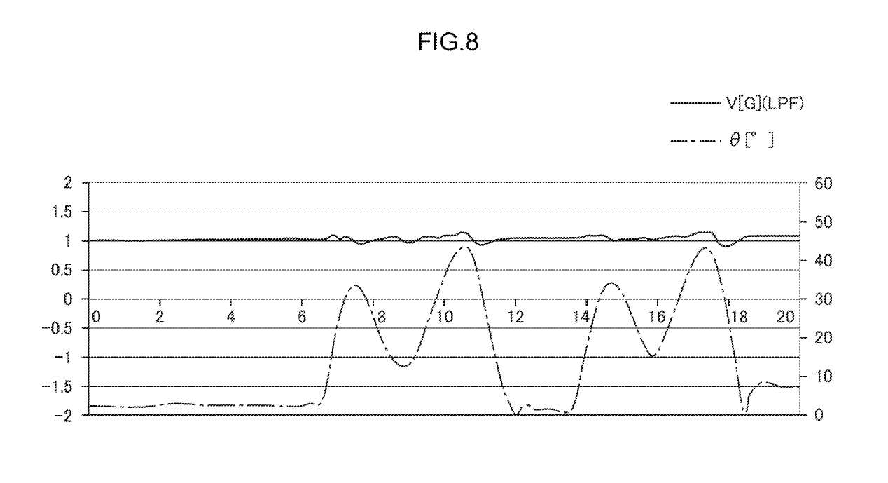

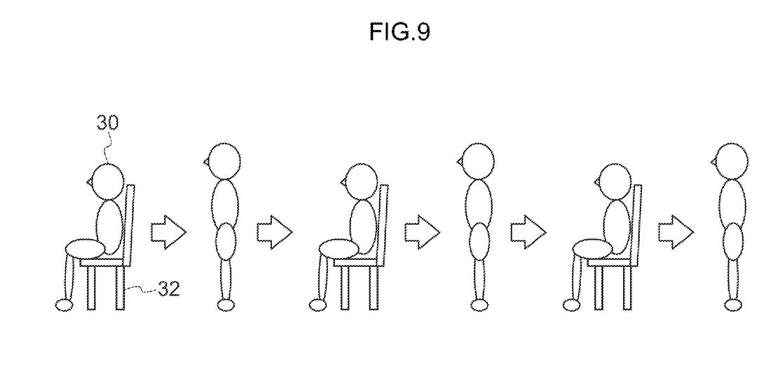

FIG. 7 and FIG. 8 illustrate examples of waveforms of the composite acceleration V and the trunk inclination angle θ when the standing up action is performed three times as illustrated in FIG. 9. FIG. 7 illustrates waveforms of the composite acceleration and the trunk inclination angle of a 69-year-old male without fear of fall and physical pains. FIG. 8 illustrates waveforms of the composite acceleration and the trunk inclination angle of an 84-year-old female who frequently uses a walking stick and has trouble standing up.

In the case of the 69-year-old male without fear of fall and physical pains, as illustrated in FIG. 7, the waveforms of the composite acceleration V and the trunk inclination angle θ rise and fall in a clear-cut manner, and it is conceivable that there are no particular problems regarding motor function.

On the other hand, in the case of the 84-year-old female who frequently uses a walking stick and has trouble standing up, as illustrated in FIG. 8, the composite acceleration V hardly varies while the trunk inclination angle θ slowly varies to a great extent. This suggests that the standing up action is performed fairly slowly inclining her trunk and the action of standing up from a chair is fairly difficult for her.

At step S114, a motor function index MF is calculated based on the strength score PO calculated at step S110 and the angle score TR calculated at step S112, using the following equation.

MF=a3×PO+b3×TR+c3 (8)

Here, a3, b3, and c3 are coefficients determined by using such a technique as principle component analysis or linear regression, for example, on measurement results of the composite acceleration V for several subjects performing a standing up action from a seated state in a chair, and are set to values such that the higher is the motor function of the user, the higher is the motor function index MF. The motor function index MF calculated in this manner using Equation (8), using the strength score PO and the angle score TR as parameters, can be said to be a motor function evaluation value representing motor functions, including the muscle strength and the standing balance performance, of the user 30.

At step S116, the strength score PO, the angle score TR, and the motor function index MF calculated at step S110 to step S114, are output to the display section 16 and displayed. The strength score PO, the angle score TR, and the motor function index MF are also associated with the current date and time acquired from the clock section 20, and are output to and stored in the non-volatile memory 12D.

As an example in the present exemplary embodiment, the strength score PO, the angle score TR, and the motor function index MF are converted to deviation values and then displayed. In the computation of the deviation values, for example, the computation equations for the deviation values may each be derived based on test results by which the strength score PO, the angle score TR and the motor function index MF were determined for several subjects and be stored in the non-volatile memory 12D in advance. And, the deviation values for the strength score PO, the angle score TR, and the motor function index MF may be calculated using each computation equation stored in the non-volatile memory 12D.

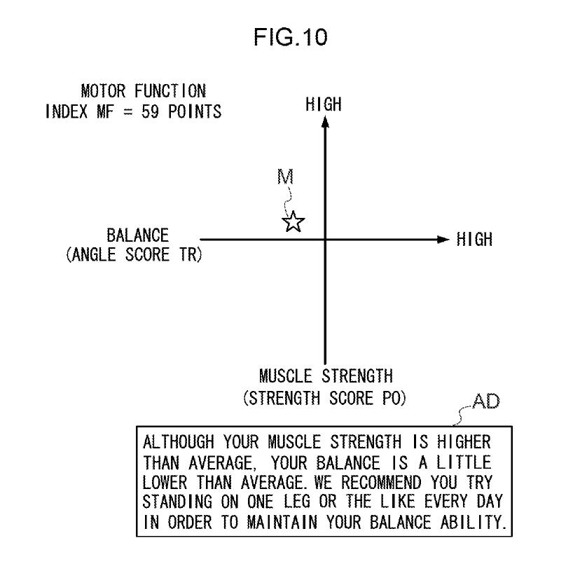

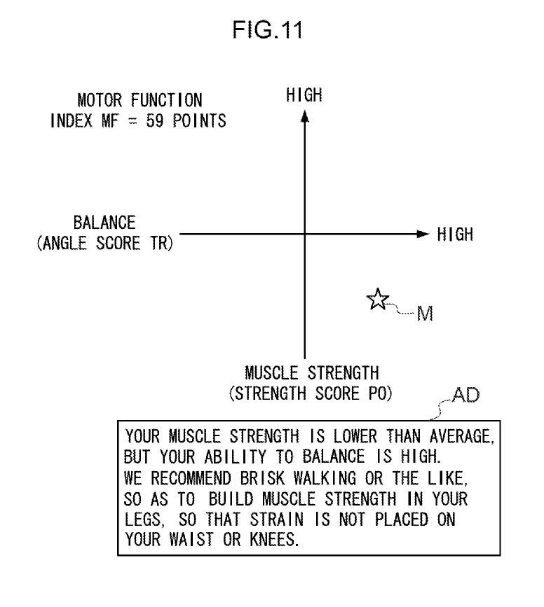

FIG. 10 and FIG. 11 illustrate display examples for each score. In FIG. 10 and FIG. 11, the strength score PO, the angle score TR, and the motor function index MF are represented with the respective deviation values. As illustrated in FIG. 10 and FIG. 11, the vertical axis corresponds to the strength score PO indicating the muscle strength of the user, the horizontal axis corresponds to the angle score TR indicating the standing balance ability of the user, and a mark M is plotted and displayed at a position corresponding to the strength score PO and the angle score TR calculated at step S110 and step S112, respectively. An exemplary case where the deviation values of the strength score PO and the angle score TR are both 50 at the point of intersection between the horizontal axis and the vertical axis is illustrated. Moreover, the motor function index MF and advice data AD are also displayed.

In FIG. 10 and FIG. 11, if the strength score PO is higher, then the mark M is plotted further to the upper side, if the angle score TR is higher, then and the mark M is plotted further to the right.

The advice data AD is advice data corresponding to the strength score PO and the angle score TR. For example, advice data table data representing correspondence relationships among the strength score PO, the angle score TR, and the advice data AD may be stored in the non-volatile memory 12D in advance, and the advice data AD corresponding to the strength score PO and the angle score TR calculated at step S110 and at step S112, respectively, may be acquired from the advice data table data and be displayed on the display section 16.

FIG. 10 and FIG. 11 both illustrate examples of displays, in which the motor function index MF is 59, as an example. As illustrated in FIG. 10 and FIG. 11, the strength score PO and the angle score TR sometimes differ even though the motor function index MF is the same, and in such cases, the contents of the advice data AD are different. Since the advice data AD includes data such as what type of training should be performed to improve the current situation, not only can the user ascertain the current state of their motor functions, but also the user can easily ascertain what type of training should be performed to improve their motor functions.

A motor function age MFA may be calculated as the motor function evaluation score of the user using the following equation and displayed on the display section 16.

MFA=(a4×PO+b4×TR+c4×AG)×d+e (9)

Here, AG is the actual age of the user 30, and the user 30 may be prompted to input their actual age, by displaying a screen image on the display section 16 for the user 30 to input actual age before measurement initiation. Moreover, inputs a4, b4, c4, d, and e are coefficients determined by using such a technique as principle component analysis or linear regression, for example, on measurement results of the composite acceleration V for several subjects performing a standing up action from a seated state in a chair, and are set to values such that the higher is the motor functions of the user 30, the lower is the motor function age MFA.

By computing and displaying a motor function age MFA based on the actual age in this manner, the user can easily ascertain roughly what age his/her motor function age corresponds to, and a motivation to maintain and improve muscle strength and balance ability can be promoted.

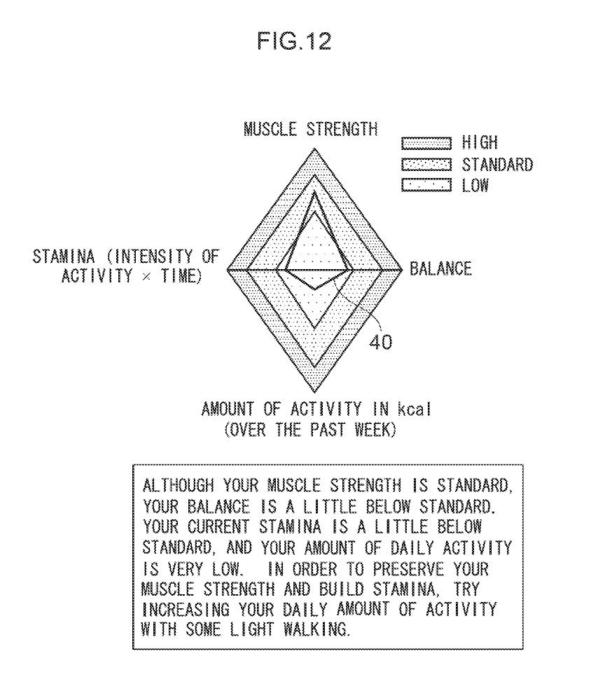

The motor function measurement apparatus 10 may be installed in an activity monitor capable of measuring, for example, an activity level, stamina (intensity of activity×time) and the like of the user. In such a case, activity level and stamina measured by the activity monitor may be displayed on the display section 16 as motor function evaluation values, besides the muscle strength (strength score PO), and the balance ability (angle score TR). FIG. 12 illustrates an example of such a display. In the example shown in FIG. 12, the activity level is displayed as a cumulative amount (kcal) of activity level for the past one week. As illustrated in FIG. 12, it can easily be ascertained what level each motor function evaluation value corresponds to, among three grades: “low”, “standard”, and “high”, by using the shape of a rhombus 40. Which motor function evaluation values need to be improved can thereby be ascertained easily.

As illustrated in FIG. 12, the advice data AD corresponding to the muscle strength, the balance ability, the activity level and the stamina may be displayed on the display section 16. For example, advice data table data representing correspondence relationships between the advice data AD and muscle strength, balance ability, activity level and stamina, may be stored in the non-volatile memory 12D in advance, and the advice data AD corresponding to the muscle strength, balance ability, activity level and stamina may be acquired from this advice data table data and displayed on the display section 16.

In this manner, in the present exemplary embodiment, the strength score PO relating to the muscle strength of the user, the angle score TR relating to the balance ability of the user, and the motor function index MF are calculated as motor function evaluation values, as motor function evaluation values based on the acceleration measured when the user performs a standing up action from a chair, and advice data corresponding thereto is displayed. The exemplary advantageous effects listed below can thereby be achieved.
Motor functions can be measured simply and safely, within a short time.
Measurements can be made at any time, every day, without taking a lot of time, since measurements can be made by oneself
There is no need to perform various physical strength tests such as walking or muscle strength measurements, and no specialist knowledge is required.
As long as there is a chair or any substitute thereof and enough space to stand up, there are no limitations on the measurement location. For example, if a constant motor function measurement mode is provided, measurements can be made while a user is unaware since motor functions can be measured when performing a standing up action, for example, after finishing using a western-style toilet.
Measurement is possible even if the user has trouble walking, as long as they can stand up and sit down.
Making periodic measurements enables deterioration in motor function to be detected early, physical conditions such as degree of pain in the waist or knees to be ascertained, bedridden states to be prevented or the like, and the apparatus can be used effectively in a healthcare facility, a rehabilitation center, or the like.
Since confidence can be gained if the motor function evaluation value is high, the frequency of outings can be increased, and quality of life (QOL) can be improved.
If motor function evaluation values are maintained over time, the user can feel relieved. On the other hand, if the motor function evaluation values are decreasing, effort can be made to increase motor function evaluation values by exercising or the like. Thus, the user's ability to walk can be expected to be maintained or improved.

Although explanation has been given in the present exemplary embodiment regarding an exemplary case where the motor function index MF is calculated based on the strength score PO and the angle score TR, the motor function index MF may be calculated using the time taken to stand up, daily activity levels, and the like as parameters.

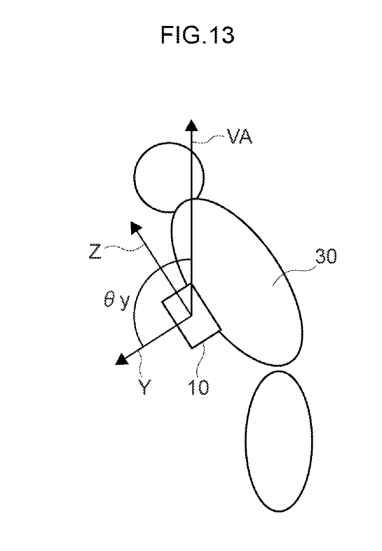

Although explanation has been given in the present exemplary embodiment regarding an exemplary case where the inclination angle θz of the z-axis, calculated using Equation (4), is calculated as the trunk inclination angle θ of the user, the inclination angle θy of the y-axis, calculated using Equation (3), may be calculated as the trunk inclination angle of the user when the motor function measurement apparatus 10 is worn as illustrated in FIG. 2, such that the i-axis is along the upright direction, the y-axis is along the front-rear direction of the user, and the x-axis is in the left-right direction of the user when the user is upright. If the user 30 performs a standing up action from a chair with their trunk inclined in the front-rear direction as illustrated in FIG. 13, the y-axis inclines with respect to the vertical axis VA when the motor function measurement apparatus 10 is worn such that the y-axis is along the front-rear direction of the user, and the inclination angle θy can be said to indicate the front-rear direction incline of the trunk.

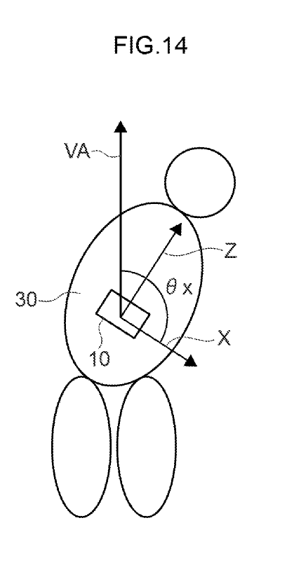

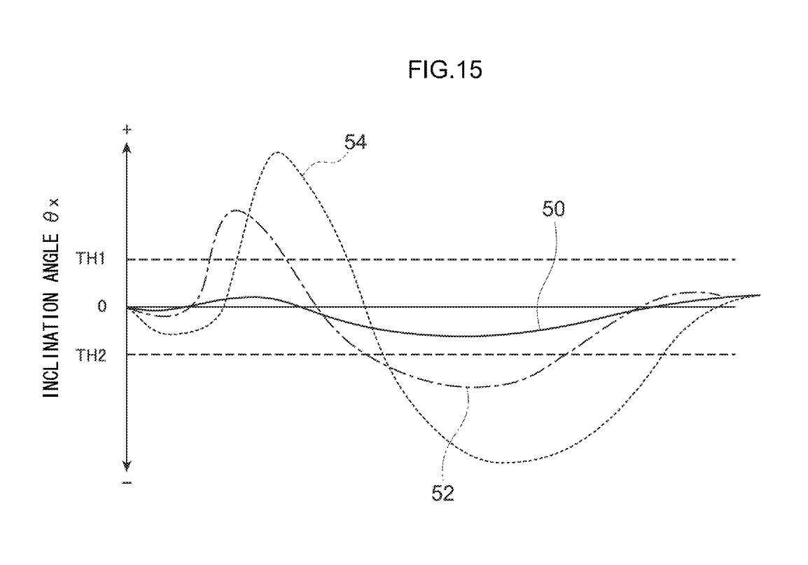

As illustrated in FIG. 14, if the user 30 performs a standing up action from the chair and inclines their trunk in the left-right direction, the x-axis inclines with respect to the vertical axis VA, and the inclination angle θx, calculated using Equation (2), can be said to indicate the left-right direction inclination of the trunk. It is conceivable that inclination of the trunk toward either the left or the right side when the user stands up from a chair, occurs due to pain arising on either the left side or the right side of the body. For example, when intense pain arises in the right knee, a large force will naturally be placed on the left knee when standing up from a chair so as to avoid placing force on the right knee, and the body inclines to the left side. FIG. 15 illustrates examples of a waveform 50 of the inclination angle θx when there is pain in none of knees, a waveform 52 of the inclination angle θx when there is a little pain in the right knee, and a waveform 54 of the inclination angle θx when there is intense pain in the right knee. Note that, in these examples, a positive value of the inclination angle θx indicates inclination of the body to the left side, and a negative value of the inclination angle θx indicates inclination of the body toward the right side.

As illustrated in FIG. 15, the waveform 50 for the exemplary case where there is no pain in either knee, exhibits little variation, and there is little inclination of the body toward the left-right direction. In the waveform 52 for the exemplary case where there is a little pain in the right knee, the value of the inclination angle θx initially varies in the positive direction since the body inclines to the left side when standing up from the chair so as not to place force on the right knee. Then when the action of standing up is complete, the value of the inclination angle θx varies in the negative direction since the body inclines to the right side as a response to the body inclining to the left side while standing up. In the waveform 54 for the exemplary case where there is intense pain in the right knee, the inclination angle θx varies to a greater extent than that in the waveform 52 for the exemplary case where in which there is a little pain in the right knee. In this manner, the inclination angle θx increases as the pain in the knee intensifies.

Determination may be made as to whether pain arises in either the left side or the right side of the user based on the inclination angle θx measured during the measurement period. For example, when the inclination angle θx of the first peak appearing in the waveform of the inclination angle θx is a predetermined positive threshold value TH1 or greater, it is determined that there is pain arising in the right side of the body since the body inclines to the left side, and when the inclination angle θx of the first peak appearing in the waveform of the inclination angle θx is a predetermined negative threshold value TH2 or less, it is determined that there is pain arising in the left side of the body since the body inclines to the right side.

Determination that pain is arising in either the left or right side of the body can accordingly be made based on the inclination angle θx in the left-right direction.

Although explanation has been given in the present exemplary embodiment regarding an exemplary case where the trunk inclination angle is calculated based on acceleration, the trunk inclination angle may be directly detected using a gyrosensor.

Although explanation has been given in the present exemplary embodiment regarding an exemplary case where motor function evaluation values are measured by performing an action of standing up from a chair as a predetermined action, the standing up can be performed from anything other than chair and the predetermined action is not limited to standing up. It is sufficient that the predetermined action is an action in which at least a part of the body moves, such as the action of sitting on a chair from a standing state, the action of standing from a seated state on the floor, the action of sitting on the floor from a standing state, the action of raising one's upper body from a lying-down state, the action of lying down from a state in which one's upper body is risen, the action of climbing stairs, the action of descending stairs, or the like.

Although explanation has been given in the present exemplary embodiment regarding an exemplary case where the motor function measurement apparatus 10 is a portable and dedicated device, such a configuration that a personal computer 80 is connect, by wire or wireless, to a measurement device 82 including the measurement section 14 as illustrated in FIG. 13, for example. In such cases, the personal computer 80 functions as a motor function measurement apparatus by acquiring acceleration measured along each axis by the measurement device 82, and executing the processing illustrated in FIG. 3. Moreover, it is not required to be limited to a personal computer; the measurement device 82 may be connected, by wire or wireless, to a portable terminal such as a mobile phone, a smart phone, a tablet terminal, or the like, and the portable terminal may be caused to function as the motor function measurement apparatus.

What is claimed is:

1. A motor function measurement apparatus, comprising:
a measurement data acquisition section that acquires measurement data including at least one of an acceleration or a trunk inclination angle of a user, the measurement data being measured during a measurement period in which the user performs a predetermined action; and
a motor function evaluation value computation section that, based on the measurement data, calculates a motor function evaluation value related to a motor function of the user.

2. The motor function measurement apparatus of claim 1, wherein:
the measurement data includes the acceleration and the trunk inclination angle; and
the motor function evaluation value computation section calculates, based on the acceleration and the trunk inclination angle of the user measured during the measurement period, a motor function index related to muscle strength and balance ability of the user as the motor function evaluation value.

3. The motor function measurement apparatus of claim 1, further comprising
an age acquisition section that acquires age data related to an age of the user, wherein
the motor function evaluation value computation section calculates, based on the acceleration, the trunk inclination angle and the age data, a motor function age related to muscle strength and balance ability of the user as the motor function evaluation value.

4. The motor function measurement apparatus of claim 1, wherein:
the measurement data includes the acceleration; and
the motor function evaluation value computation section calculates, based on the acceleration of the user measured during the measurement period, a strength score related to muscle strength of the user as the motor function evaluation value.

5. The motor function measurement apparatus of claim 4, wherein:
the motor function evaluation value computation section calculates, based on a maximum value of acceleration of the user measured during the measurement period, a difference between a minimum value and the maximum value of acceleration of the user measured during the measurement period, and a maximum rate of change in the acceleration of the user measured during the measurement period, a strength score related to the muscle strength of the user as the motor function evaluation value.

6. The motor function measurement apparatus of claim 1, wherein:
the measurement data includes the trunk inclination angle; and
the motor function evaluation value computation section calculates, based on the trunk inclination angle of the user measured during the measurement period an angle score related to a balance ability of the user as the motor function evaluation value.

7. The motor function measurement apparatus of claim 6, wherein:
the motor function evaluation value computation section calculates, based on a difference between a maximum value of the trunk inclination angle of the user measured during the measurement period and a reference trunk inclination angle that is a trunk angle of the user measured during a rest period of the measurement period in which the user is in a resting state, and based on a number of peaks in the trunk inclination angle of the user measured during the measurement period, an angle score related to the balance ability of the user as the motor function evaluation value.

8. The motor function measurement apparatus of claim 1, wherein:
the measurement data includes the trunk inclination angle of the user in a left-right direction of the user; and
the motor function measurement apparatus further comprises a determination section that determines whether or not pain arises at either of a left side or a right side of a body of the user based on the trunk inclination angle of the user in the left-right direction measured during the measurement period.

9. The motor function measurement apparatus of claim 1, further comprising an output section that outputs the motor function evaluation value, and outputs advice data corresponding to the motor function evaluation value.

10. The motor function measurement apparatus of claim 1, wherein the predetermined action is an action of standing up from a seated state in a chair.

11. The motor function measurement apparatus of claim 1, further comprising a measurement section that measures at least one of the acceleration or the trunk inclination angle.

12. A motor function measurement method, comprising:
acquiring measurement data including at least one of an acceleration or a trunk inclination angle of a user, the measurement data being measured during a measurement period in which the user performs a predetermined action; and
based on the measurement data, computing a motor function evaluation value related to a motor function of the user.

13. A non-transitory recording medium storing a motor function measurement program that causes processing to be executed on a computer, the process comprising:
acquiring measurement data including at least one of an acceleration or a trunk inclination angle of a user, the measurement data being measured during a measurement period in which the user performs a predetermined action; and
based on the measurement data, computing a motor function evaluation value related to a motor function of the user.
